(19) United States
(12) Patent Application Publication
(10) Pub. No.: US 2015/0053212 A1
(43) Pub. Date: Feb. 26, 2015

(54) JAW THRUST APPARATUS

(71) Applicant: Phillip Guerra, Arlington, TX (US)

(72) Inventor: Phillip Guerra, Arlington, TX (US)

(21) Appl. No.: 13/970,813

(22) Filed: Aug. 20, 2013

(51) Int. Cl. A61F 5/37

(52) U.S. Cl. CPC A61F 5/3707
USPC 128/869

(57) ABSTRACT

An apparatus and method to apply bilateral medial pressure against a position proximate to the ramus of the mandible jawbone of a patient. The apparatus includes a first pressure device, a second pressure device, and a locking mechanism operably associated with the first pressure device and the second pressure device. The method includes simultaneously applying bilateral medial pressure against a position proximate to the ramus of the mandible jawbone of the patient and locking the first pressure devices in position with the locking mechanism.

### BACKGROUND

1. Field of the Invention

The present application relates generally to medical devices, and more specifically, to a jaw thrust apparatus and method of use.

2. Description of Related Art

Method and devices configured to retain the airway in an open position is well known in the art. During medical procedures, it is critical that the patient's airway remains open for proper breathing during operation. It is especially important in procedures where anesthesia is used because the anesthesia causes the muscles to relax and renders the patient in a sedated state, which in turn prevents the patient from waking up if the airway passage is blocked.

A common method to maintain an open airway includes the process of inserting an endotracheal tube through the mouth. However, endotracheal tubes can cause irritation of the throat and/or vocal chords that could lead to additional surgeries or problems.

Another commonly known method includes the process of having the anesthesiologist monitor and continuously hold the patient's head in the proper position. If the airway becomes blocked, the anesthesiologist can merely perform a chin lift maneuver by tilting and holding the patients chin in a backward fashion, or a “jaw thrust” by pushing the patient's jaw upward to open the airway. However, this method disadvantageously requires the anesthesiologist to continuously monitor and hold the patient's head throughout the procedure, thereby limiting the tasks the anesthesiologist can do. Furthermore, during long procedures, the anesthesiologist may become fatigued an incapable of effectively performing the task.

Yet another method includes using an adjustable air management device that rests under the head of the patient and applies pressure at selected areas on the jaw. The pressure applied against opens the jaw, which in turn maintains an open airway. U.S. Pat. No. 7,055,524 to Taimoorazy is one known embodiment indicative of a current state-of-the-art air management device.

FIG. 1 depicts a side view of the Taimoorazy device 101 having a base 103 adjustably attached to a post 105 and a padded head 107. The Taimoorazy device 101 applies a force F1 on the mandibular angle of the jaw, as depicted with arrow F1. The applied force F1 causes the jaw to pivot open, which in turn maintains an open airway. It should be understood that the Taimoorazy device 101 is not configured to apply bilateral medial pressure against the laryngospasm notches of the patient (hereinafter shown in phantom).

Although the Taimoorazy device 101 provides effective means for opening the jaw and retaining an open airway, considerable shortcomings remain. For example, extensive time and effort is exhausted preparing device 101 prior to and during use. In addition, the device 101 is configured to retain the patient's head in a neutral, non-angled position, which is not always ideal in scenarios, e.g., wherein the patients head must rest at a slightly angled position. Furthermore, the device 101 must be placed underneath the patient's head, thereby requiring the potentially dangerous process of lifting the head prior to use. It should be understood that in some scenarios, lifting the head is an undesired process that can be dangerous, e.g., neck injuries.

### DESCRIPTION OF THE DRAWINGS

The novel features believed characteristic of the invention are set forth in the appended claims. However, the invention itself, as well as a preferred mode of use, and further objectives and advantages thereof, will best be understood by reference to the following detailed description when read in conjunction with the accompanying drawings, wherein:

FIG. 1 is a side view of a conventional airway management device;

FIG. 2 is a top view of a jaw thrust apparatus according to a preferred embodiment of the present application;

FIG. 3 is a top view of an alternative embodiment of a pressure device;

FIG. 4 is a top view of the jaw trust apparatus of FIG. 2;

FIG. 5 is a top, partially cutout view of the pressure device of the jaw trust apparatus of FIG. 4; and

FIG. 6 is a side view of the jaw trust apparatus according to an alternative embodiment of the present application.

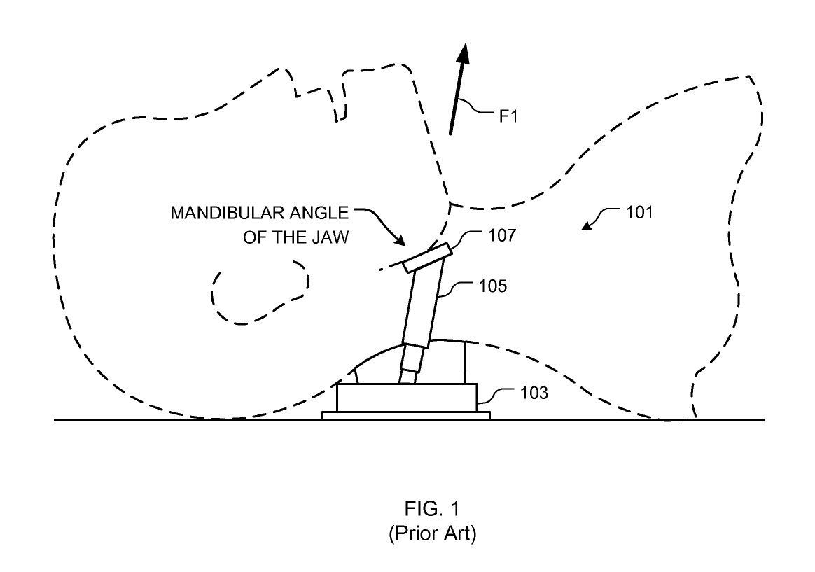

While the apparatus and method according to the present application are susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the process of the present application as defined by the appended claims.

### DETAILED DESCRIPTION OF THE PREFERRED EMBODIMENT

Illustrative embodiments of the jaw trust apparatus and methods of use are provided below. It will of course be appreciated that in the development of any actual embodiment, numerous implementation-specific decisions will be made to achieve the developer's specific goals, such as compliance with assembly-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, shall refer to this application as a whole and not to any particular portions of this application. When the claims use the word “or” in reference to a list of two or more items, that word covers all of the following interpretations of the word: any of the items in the list, all of the items in the list and any combination of the items in the list.

The jaw trust apparatus of the present application overcomes the abovementioned problems commonly associated with conventional systems and methods to maintain an open airway of a sedated patient. Specifically, the present invention is configured to open the airway by applying bilateral medial pressure at a position to the posterior to the ramus of the mandible of a jaw and anterior to the mastoid processes, which in turn forces the jaw and airway to open. The jaw trust apparatus is also provided with a locking mechanism configured to lock the apparatus in a fixed position during use. It will be appreciated that the jaw thrust apparatus has proven to be an effective, rapid, and cost-reducing means to retain the airway in an open position, thereby greatly reducing the time exhausted in the time-sensitive and potentially life-threatening procedure. Further detailed description and illustration of the different embodiments and methods are provided below and shown in the corresponding figures.

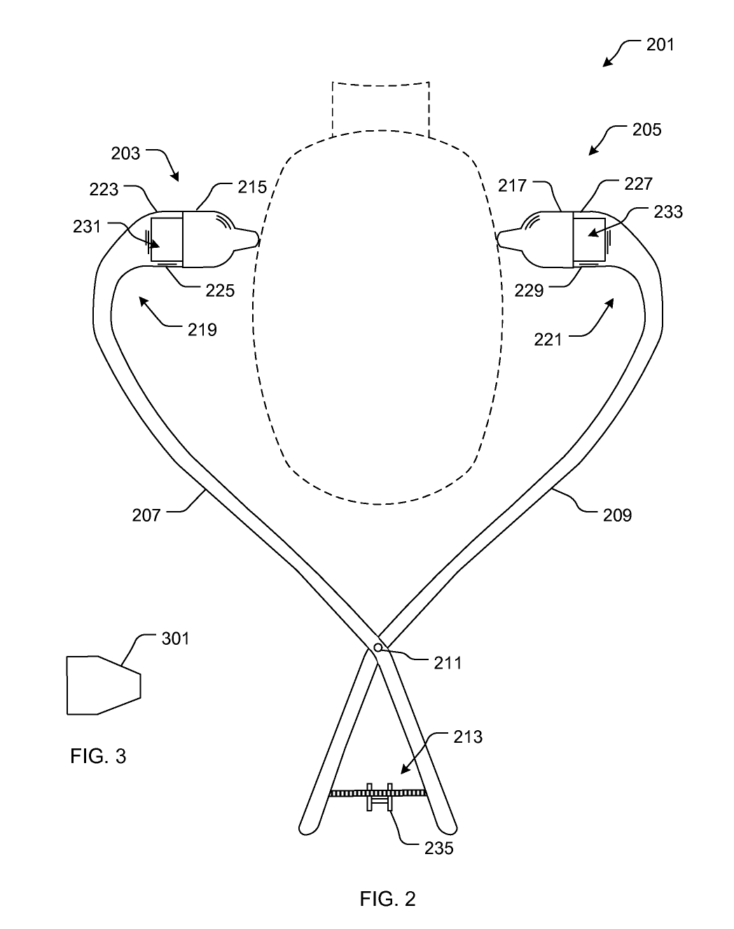

Referring now to the drawings wherein like reference characters identify corresponding or similar elements throughout the several views, FIG. 2-6 depicts various views of a jaw thrust apparatus 201 in accordance with a preferred embodiment of the present application. Apparatus 201 is configured to apply and retain bilateral medial pressure against the posterior to the ramus of the mandible of the jaw and anterior to the mastoid processes. For example, as depicted in the figures, the jaw thrust device is configured to apply bilateral medial pressure against the laryngospasm notches of the patient during operation. Hereinafter, the figures depict the jaw trust device operably associated with the laryngospasm notches; however, it will be appreciated that the apparatus 201 could be operably associated with other positions posterior to the ramus of the mandible and anterior to the mastoid process.

It should be understood that the jaw thrust configured to apply bilateral medial pressure posterior to the ramus of the mandible and anterior to the mastoid processes causes anterior motion of the mandible thus relieving obstruction from the tongue falling back or other soft tissue obstruction in the pharynx. The laryngospasm notch is a very closely related anatomical location, but by applying bilateral medial pressure here, the painful stimulation supposedly allows the user to break a spasming larynx (i.e. closed vocal cords preventing airflow) which can be different from applying bilateral medial pressure to the posterior to the ramus of the mandible and anterior to the mastoid processes that causes the mandible to move anteriorly.

FIG. 2 depicts a top view of apparatus 201 having two pressure devices 203, 205 attached to respective arms 207, 209. The pressure devices are configured to come into contact with the laryngospasm notches, while the arms are configured to retain the pressure devices in a relatively locked position. Arms 207, 209 pivotally attach about a pivot joint 211 and are secured in a locked position via a locking mechanism 213.

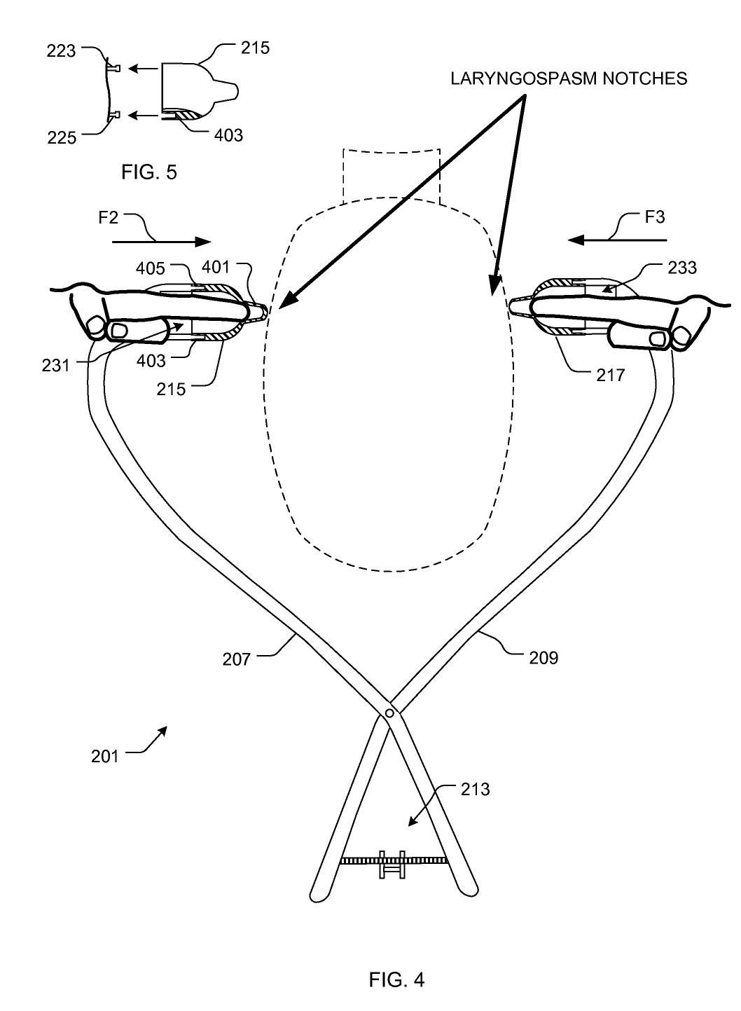

In the contemplated embodiment, pressure devices 203, 205 are composed of a durable, elastomeric material having a nipple-shaped geometry. It will be appreciated that the nipple-shaped geometry provides efficient means to receive the index finger of a user (e.g., see FIG. 4). One feature believed characteristic of the present invention is removably attaching the pressure device, e.g., nipples 215, 217 from the arms, which in turn facilitates easy and rapid disposal of the pressure device after use.

Arms 207, 209 preferably bifurcate at respective endings 219, 221 to form a plurality of attachment members 223, 225, 227, and 229. The attachment members are configured to receive and interlock with the pressure devices. In the preferred embodiment, the attachment members are separated from each other to form gaps 231, 233. The gaps are sufficiently spaced apart from each other so as to receive the index fingers of the user therethrough (see, e.g., FIG. 4).

Locking mechanism 213 provides efficient means for locking the arms as bilateral medial force is applied via pressure devices 203, 205. In one contemplated embodiment, locking mechanism is a device that gradually ratchets into position with movement of the arms and is configured to be release from the locked position via a quick release device 235. Other locking mechanisms having these features are also contemplated in alterative embodiments.

It will be appreciated that alternative embodiments of pressure devices could include wedges and/or other geometric shapes and sizes in lieu of the preferred nipple-shaped configuration. For example, FIG. 3 illustrates a top view of an alternative embodiment, wherein the pressure device 301 is manufactured having a wedge-shaped configuration.

In FIGS. 4 and 5, top views of apparatus 201 are depicted. For simplicity, only one of pressure devices and associated attachment members is discussed in detail, although both pressure devices share the same features.

Nipple 215 is composed of an elastomeric material that stretches and conforms to the index finger. In the contemplated embodiment, the nipple has an ending 401 that elastically deforms and receives the tip of the index finger. This feature allows the user to accurately position the nipple on the laryngospasm notch with the index fingers. During operation, the user selectively positions apparatus 201 so that the nipples 215, 217 come in contact with the laryngospasm notches of the patient, then applies bilateral medial pressure against that notches with the index fingers, as indicated with arrow F2 and F3. The locking mechanism 213 repeatively locks “ratchets” arms 207, 209 into a fixed position relative to each other as forces F2, F3 are applied.

As discussed, the nipples are removably attached to the attachment members. To facilitate this feature, nipple 215 is provided with one or more notches 403, 405 or similar means to receive and interlock with attachment members 223, 225. The attachment members are shaped to receive and secure to the nipples.

These above-described features of apparatus 201 provide significant improvements over the known art. For example, the apparatus allows for jaw thrust maneuverability that can mimic the motion an anesthesiologist would manually perform in such procedures. The apparatus can be utilized such that only medial pressure is applied at selective locations, thus eliminating the need to use upward pressure. Further, the apparatus 201 can be utilized when the patient's head is in a non-neutral, non-zero angel. For example, in some scenarios, the head will be rotated slightly, thereby causing difficulty in utilizing known devices. The apparatus also does not require the potentially dangerous process of lifting the patient's head.

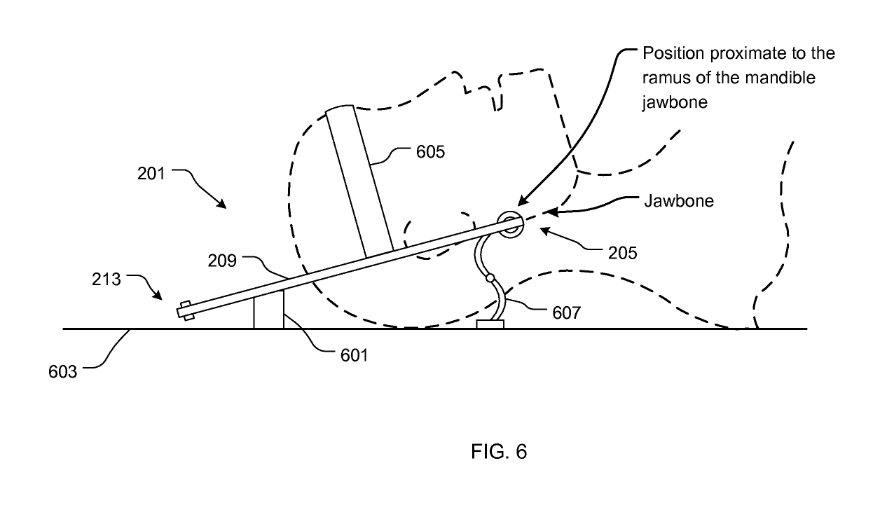

FIG. 6 shows a side view of apparatus 201 with additional optional features. In the exemplary embodiment, apparatus 201 is further provided with a stand 601 configured to support arms 207, 209 in a fixed position relative to a resting structure 603. The stand can be removably attached to either one or both of arms 207. Apparatus 201 could also include one or more straps 605 attached to arms 207, 209 and/or pressure devices 203, 205. In the contemplated embodiment, the strap is configured to extend over or under the head of the patient, e.g., the forehead or face. Also, one of more adjustable supports 607 could be utilized to support either ending 219 and/or ending 221 in a fixed position relative to resting structure 603.

Particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless the above Detailed Description section explicitly defines such terms.

Accordingly, the actual scope of the invention encompasses not only the disclosed embodiments, but also all equivalent ways of practicing or implementing the invention.

The above detailed description of the embodiments of the invention is not intended to be exhaustive or to limit the invention to the precise form disclosed above or to the particular field of usage mentioned in this disclosure. While specific embodiments of, and examples for, the invention are described above for illustrative purposes, various equivalent modifications are possible within the scope of the invention, as those skilled in the relevant art will recognize. Also, the teachings of the invention provided herein can be applied to other systems, not necessarily the system described above. The elements and acts of the various embodiments described above can be combined to provide further embodiments.

Changes can be made to the invention in light of the above “Detailed Description.” While the above description details certain embodiments of the invention and describes the best mode contemplated, no matter how detailed the above appears in text, the invention can be practiced in many ways. Therefore, implementation details may vary considerably while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated.

While certain aspects of the invention are presented below in certain claim forms, the inventor contemplates the various aspects of the invention in any number of claim forms. Accordingly, the inventor reserves the right to add additional claims after filing the application to pursue such additional claim forms for other aspects of the invention.

What is claimed is:

1. A jaw thrust apparatus configured to apply bilateral medial pressure at a position posterior to the ramus of the mandible jawbone, the apparatus comprising:
a first pressure device;
a second pressure device; and
a locking mechanism operably associated with the first pressure device and the second pressure device;
wherein the first pressure device and the second pressure device are configured to move towards each other with force applied thereagainst via a user and configured to come into contact at a position proximate to the ramus of the mandible jawbone, which in turn causes an airway of the patient to open; and
wherein the locking mechanism is configured to lock the first pressure device and the second pressure device in a relatively locked position so as to maintain the airway in an open position by apply pressure against the position proximate to the ramus of the mandible jawbone.

2. The apparatus of claim 1, further comprising:
a first arm associated with the first pressure device; and
a second arm associated with the second pressure device;
wherein the first arm and the second arm pivot relative to each other about a pivot joint.

3. The apparatus of claim 2, the first arm having:
an attachment member configured to receive the first pressure device.

4. The apparatus of claim 3, wherein the first pressure device is removable attached to the attachment member.

5. The apparatus of claim 4, the first pressure device having:
a notch configured to receive the attachment member.

6. The apparatus of claim 2, the first arm having:
a first attachment member; and
a second attachment member;
wherein the first attachment member and the second attachment member are configured to interlock with the first pressure device; and
wherein the first attachment member and the second attachment member are spaced apart relative to each other so as to form a gap.

7. The apparatus of claim 6, wherein the first pressure device is removable attached to the first attachment member and the second attachment member.

8. The apparatus of claim 3, further comprising:
a support associated with the attachment member;
wherein the support is configured to retain the attachment member in a fixed position relative to a resting structure.

9. The apparatus of claim 2, further comprising:
a locking mechanism associated with the first arm and the second arm;
wherein the locking mechanism is configured to retain the first arm relative to the second arm.

10. The apparatus of claim 2, further comprising:
a stand associated with the first arm and the second arm;
wherein the stand is configured to secure the apparatus in a position relative to a resting structure.

11. The apparatus of claim 2, further comprising:
a strap associated with the first arm and the second arm;
wherein the is configured to extend over a face of a patient and configured to secure the apparatus in a relatively fixed position.

12. The apparatus of claim 1, wherein the first pressure device is composed of an elastomeric material.

13. The apparatus of claim 1, wherein the first pressure device has a wedge-shaped configuration.

14. The apparatus of claim 1, wherein the first pressure device is configured to receive an index finger of the user.

15. The apparatus of claim 1, the first pressure device having a nipple-shaped contouring configured to receive an index finger of the user;
wherein the nipple elastically stretches to receive the tip of the index finger.

16. A jaw thrust apparatus, comprising:
a first arm and a second arm pivotally attached to each other;
a first pressure device removable secured to the first arm and second pressure device removably secured to the second arm;
wherein the first arm and the second arm are configured to pivot relative to each other, which in turn causes the first device and the second device move towards each other;
wherein the first device and the second device are configured to receive index fingers of a user; and
wherein the first pressure device and the second pressure device are configured to move parallel relative to each other so as to apply bilateral medial pressure against a position proximate to the ramus of the mandible jawbone; and
wherein the bilateral medial pressure causes an airway to open.

17. A method to maintain an open airway of a sedated patient, the method comprising:
simultaneously applying bilateral medial pressure against a position proximate to a ramus of a mandible jawbone of the sedated patient with the apparatus of claim 1.

18. The method of claim 17, wherein the pressure being applied is achieved with index fingers inserted within the first pressure device and the second pressure device.

19. The method of claim 17, further comprising:
securing the apparatus with a strap configured to extend over the face of the sedated patient.

20. The method of claim 17, further comprising:
applying solely bilateral medial pressure against the position proximate to the ramus of the mandible jawbone.
